(19) United States
(12) Patent Application Publication
(10) Pub. No.: US 2021/0404795 A1
(43) Pub. Date: Dec. 30, 2021

(54) COMPRESSION STATE MEASURING METHOD AND COMPRESSION STATE MEASURING SYSTEM

(71) Applicant: Industrial Technology Research Institute, Hsinchu (TW)

(72) Inventors: Wan-Hsin Chen, Hsinchu City (TW); Hung-Hsien Ko, Hsinchu County (TW); Chang-Ying Chen, Hsinchu County (TW); I-Hung Chiang, Changhua County (TW)

(73) Assignee: Industrial Technology Research Institute, Hsinchu (TW)

(21) Appl. No.: 17/033,694

(22) Filed: Sep. 26, 2020

(30) Foreign Application Priority Data
Jun. 24, 2020 (TW) 109121587

(51) Int. Cl. G01B 11/16; G06T 7/00

(52) U.S. Cl. CPC G01B 11/16; G06T 2207/30124; G06T 7/001

(57) ABSTRACT

A compression state measuring method is adapted to measure a compressed state of a compressed object compressed by a compressing object. First, at least one image of a first surface region of the compressed object not covered by the compressing object is captured. A first strain distribution value of the first surface region is obtained according to the at least one image. At least one strain distribution function is obtained according to the first strain distribution value. A second strain distribution value of a second surface region of the compressed object covered by the compressing object is obtained according to the at least one strain distribution function.

### CROSS-REFERENCE TO RELATED APPLICATION

This application claims the priority benefit of Taiwan application serial no. 109121587, filed on Jun. 24, 2020. The entirety of the above-mentioned patent application is hereby incorporated by reference herein.

### BACKGROUND

### Technical Field

The disclosure relates to a measuring method and a measuring system, and also relates to a compression state measuring method and a compression state measuring system.

### Description of Related Art

With the improvement of people's standard of living, various fabric products for wearing, dressing, sitting, and lying are popularized in the consumer market. In addition to providing the basic warming or cushioning effects, these fabric products are also required to be durable. Consumers pay special attentions particularly to durability of smart wearable fabrics combining with various circuits and/or sensing device because these fabrics are more expensive than ordinary fabrics and circuits and/or sensing devices are susceptible to damage and function failure due to the stress applied thereto. Therefore, in the development of some fabric products, the strain and stress of these products under compression are required to be measured to evaluate their durability.

Nevertheless, when the strain and stress of a fabric product are measured through image capturing, an object compressing the fabric product may cover partial surface region of the fabric product. As such, the image of such region may not be captured, and the strain and stress of the region may not be obtained in real time.

### SUMMARY

An embodiment of the disclosure provides a compression state measuring method adapted to measure a compressed state of a compressed object compressed by a compressing object. First, at least one image of a first surface region of the compressed object not covered by the compressing object is captured. A first strain distribution value of the first surface region is obtained according to the at least one image. At least one strain distribution function is obtained according to the first strain distribution value. A second strain distribution value of a second surface region of the compressed object covered by the compressing object is obtained according to the at least one strain distribution function.

An embodiment of the disclosure further provides a compression state measuring system adapted to measure a compressed state of a compressed object compressed by a compressing object. The compression state measuring system includes at least one image capturing apparatus and a data processing apparatus. The image capturing apparatus is adapted to capture at least one image of a first surface region of the compressed object not covered by the compressing object. The data processing apparatus is adapted to obtain a first strain distribution value of the first surface region according to the at least one image, is adapted to obtain at least one strain distribution function according to the first strain distribution value, and is adapted to obtain a second strain distribution value of a second surface region of the compressed object covered by the compressing object according to the strain distribution function.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic view of a compression state measuring system according to an embodiment of the disclosure.

FIG. 2A and FIG. 2B illustrate a compressed object and a compressing object.

FIG. 3 is a flow chart of a compression state measuring method corresponding to the compression state measuring system of FIG. 1.

FIG. 4A and FIG. 4B illustrate measurement of the compressed object performed by a measurement apparatus of FIG. 1.

FIG. 5 is a schematic top view of the compressed object of FIG. 2B.

FIG. 6 is a curve schematically illustrating the compressed object of FIG. 5.

FIG. 7 is a schematic top view of the compressed object of FIG. 2B.

FIG. 8 and FIG. 9 are curves schematically illustrating the compressed object of FIG. 7.

FIG. 10 to FIG. 13 are schematic top views of the compressed object of FIG. 2B.

FIG. 14 is a schematic partial top view of a compressed object according to another embodiment of the disclosure.

FIG. 15 is a schematic partial top view of a compressed object according to another embodiment of the disclosure.

FIG. 16 is a curve schematically illustrating the compressed object of FIG. 15.

### DESCRIPTION OF THE EMBODIMENTS

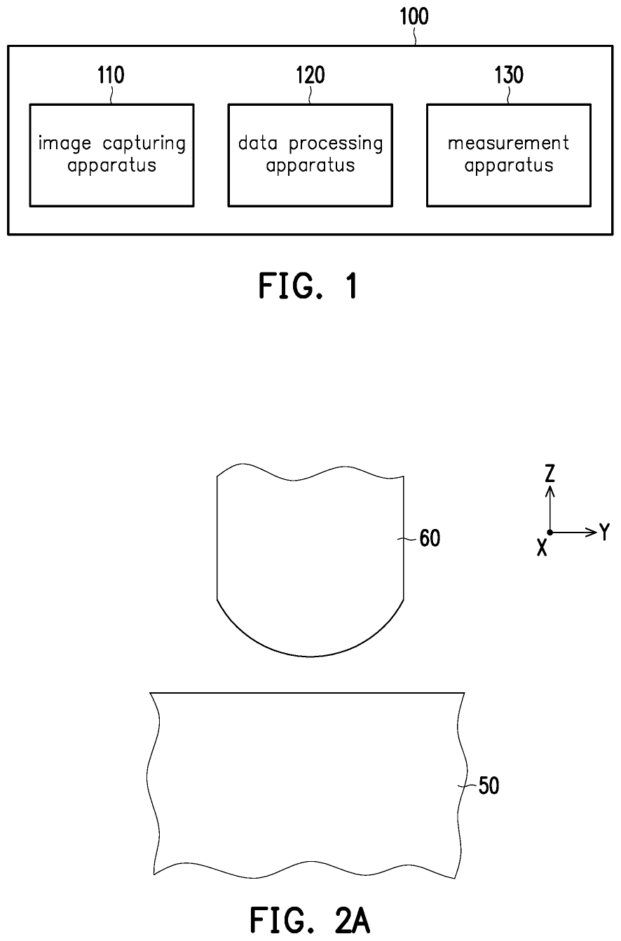

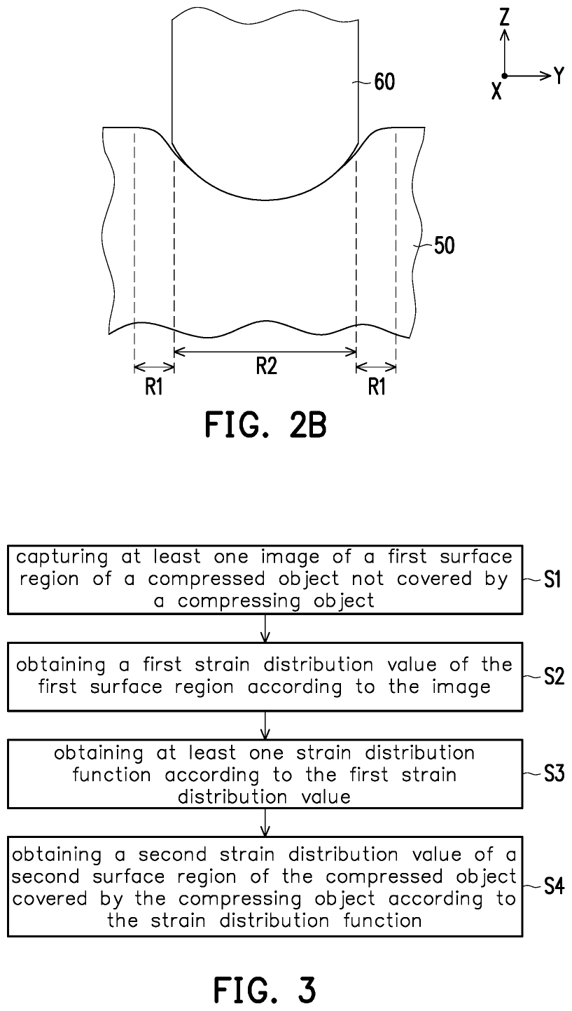

FIG. 1 is a schematic view of a compression state measuring system according to an embodiment of the disclosure. FIG. 2A and FIG. 2B illustrate a compressed object and a compressing object, where the compressed object is shown as a cross-sectional view. In FIG. 1, a compression state measuring system 100 includes at least one image capturing apparatus 110 and a data processing apparatus 120 and is adapted to measure a compressed state of a compressed object 50 shown in FIG. 2A compressed by a compressing object 60 shown in FIG. 2B. In an embodiment, hardness of the compressing object 60 is greater than hardness of the compressed object 50, and a size of the compressing object 60 is less than a size of the compressed object 50. Further, the compressed state may include a stress distribution and a strain distribution of the compressed object 50 after being compressed. The image capturing apparatus 110 may be a camera apparatus of any appropriate form, and the data processing apparatus 120 may be computer equipment and a processor thereof of any appropriate form, which is not limited by the disclosure. A measuring method of the system is described as follows.

FIG. 3 is a flow chart of a compression state measuring method corresponding to the compression state measuring system of FIG. 1. With reference to FIG. 1 to FIG. 3, first, at least one image of a first surface region R1 (shown in FIG. 2B) of the compressed object 50 not covered by the compressing object 60 is captured through the image capturing apparatus 110 (step S1). The at least one image may include images at a plurality of different time points between an uncompressed state shown in FIG. 2A and a compressed state shown in FIG. 2B. Next, the data processing apparatus 120 obtains a first strain distribution value of the first surface region R1 according to the at least one image (step S2). The first strain distribution value may include strain values of a plurality of positions in the first surface region R1. The strain values may be calculated through offset values of the positions deduced from the images at the different time points. At least one strain distribution function is obtained through the data processing apparatus 120 according to the first strain distribution value (step S3). A second strain distribution value of a second surface region R2 (shown in FIG. 2B) of the compressed object 50 covered by the compressing object 60 is obtained by the data processing apparatus 120 according to the strain distribution function (step S4).

As described above, in this embodiment, the strain distribution function corresponding to the first surface region R1 is calculated through the image of the first surface region R1 of the compressed object 50 which is not covered. Further, based on a certain degree of continuity characteristic of a deformation state of the compressed object 50 (assuming to be a homogeneous body) when being pressed, the strain distribution function may be treated as a continuous deformation state extending from the first surface region R1 to the second surface region R2. As such, in the case that the image of the covered second surface region R2 of the compressed object 50 is not obtained, the data processing apparatus 120 may calculate the strain distribution value of the second surface region R2 in real time through the strain distribution function. Further, the data processing apparatus 120 may further calculate a stress distribution value of the second surface region R2 in real time according to the second strain distribution value and an elastic modulus of the compressed object 50. To be specific, the elastic modulus of the compressed object 50 may be obtained according to the material of the compressed object 50. The product of the elastic modulus and the second strain distribution value is the corresponding stress distribution value, and the formula is P=E×ε, where P is the stress, E is the elastic modulus, and ε is the strain.

In this embodiment, the number of the image capturing apparatus 120 may be plural. In this way, images of the first surface region R1 may be captured at different angles at the same time to obtain a plurality of images corresponding to different angles of shot, and that a three-dimensional image may be constructed through optical image capturing. In addition, each of the image capturing apparatuses 120 may continuously capture the image of the first surface region R1, so that the number of the image is plural, and thus images corresponding to different time points are obtained as described above. Accordingly, the images of the first surface region R1 captured by the image capturing apparatuses 120 are complete in terms of space and time, so that the data processing apparatus 120 may accordingly calculate a detailed strain distribution function.

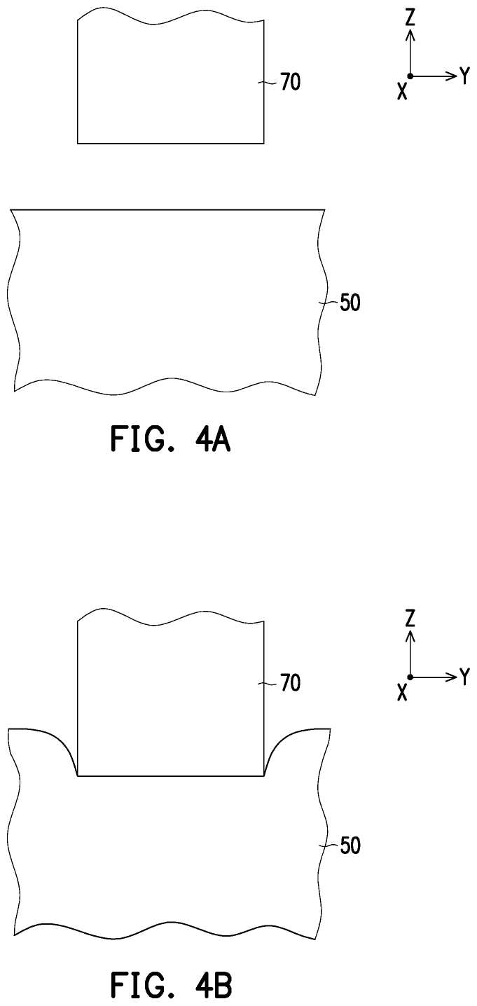

In this embodiment, the compression state measuring system 100 further includes a measurement apparatus 130 as shown in FIG. 1, and the measurement apparatus 130 is adapted to measure a maximum compressibility of the compressed object 50 within its elastic limit. FIG. 4A and FIG. 4B illustrate measurement of the compressed object performed by the measurement apparatus of FIG. 1. For instance, the compressed object 50 may be compressed through a compressing object (a compressing object 70 as shown in FIG. 4A and FIG. 4B or other appropriate compressing objects). Stresses and strains of the compressed object 50 under various different compression are respectively measured, so that a relationship curve between the stress and the strain may be accordingly established. In this way, a maximum compression rate of the compressed object 50 may be deduced through this relationship curve between the stress and the strain, and the maximum compressibility may thus be obtained. The elastic modulus (E in the foregoing formula P=E×ε) of the compressed object 50 may also be deduced through the relationship curve between the stress and the strain. Accordingly, the data processing apparatus 120 may not only calculate the second strain distribution value according to the strain distribution function as described above but may also accurately calculate the second strain distribution value depending on the maximum compressibility of the compressed object 50 at the same time.

In the foregoing embodiments, a compressing surface of the compressing object 60 is simply arc-shaped as shown in FIG. 2A and FIG. 2B, but such illustrations are merely exemplary. Actually, the compressing surface of the compressing object 60 may have various irregular shapes, and a stress distribution and a strain distribution thereof may be obtained through the measurement method provided by the compression state measuring system 100 of the present embodiment. The measurement method provided by the compression state measuring system 100 of the present embodiment is described in detailed as follows.

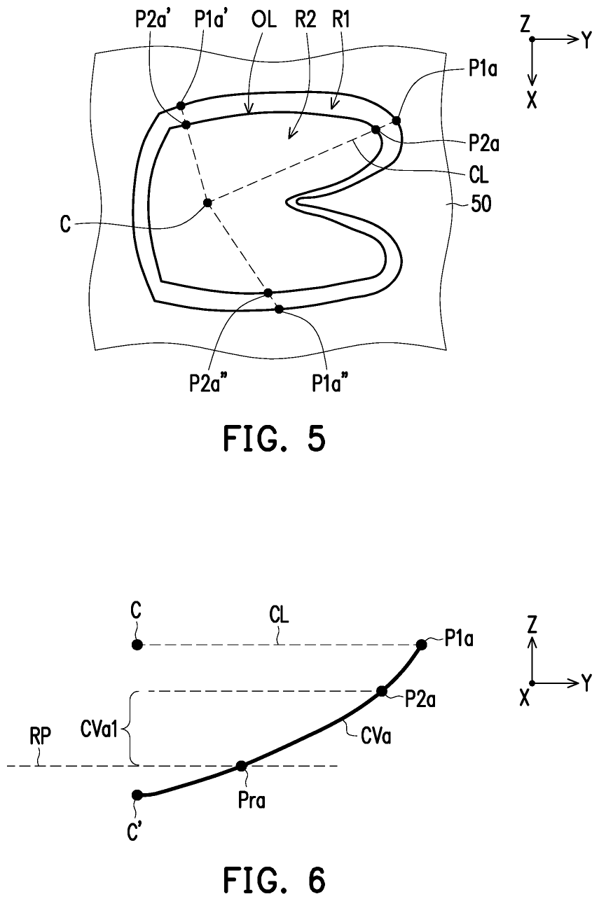

FIG. 5 is a schematic top view of the compressed object of FIG. 2B. FIG. 6 is a curve schematically illustrating the compressed object of FIG. 5. As shown in FIG. 5, a border contour OL is provided between the first surface region R1 and the second surface region R2. The data processing apparatus 120 may calculate the corresponding stress distribution value according to the first strain distribution value of the first surface region R1 and the elastic modulus of the compressed object 50 and then deduces a force center C of the compressed object 50 through such stress distribution value. Next, the data processing apparatus 120 treats any position in the first surface region R1 as a first position P1a, and a connection line CL is provided between the force center C and the first position P1a. The data processing apparatus 120 treats an intersection point of an orthogonal projection of the connection line CL in the first surface region R1 and the second surface region R2 and the border contour OL as a second position P2a.

As described above, the data processing apparatus 120 may obtain a first curve function according to a strain distribution between the first position P1a and the second position P2a in the first surface region R1. This first curve function is part of the strain distribution function. The strain distribution function corresponds to a curved surface formed by the first surface region R1, and the first curve function corresponds to a curve CVa (shown in FIG. 6) extending between the first position P1a and the second position P2a on the curved surface. In this embodiment, the first curve function is defined by a quadratic equation in two variables is, for example, but the disclosure is not limited thereto. In other embodiments, a cubic equation in two variables or a quartic equation in two variables may be used instead to define the first curve function. In addition, in other embodiments, a non-curve function may be adopted instead to replace the curve function, and disclosure is not intended to limit the function type.

As described above, as the data processing apparatus 120 treats the first curve function as a continuous deformation state extending from the first surface region R1 to the second surface region R2, a strain distribution between an orthogonal projection C′ (shown in FIG. 6) of the force center C on the second surface region R2 and the second position P2a may be calculated according to the first curve function. In this way, a plurality of different first positions P1a (two other first positions P1a′ and P1a″ and corresponding second positions P2a′ and P2a″ are schematically illustrated) are continuously selected in the first surface region R1, so that a plurality of different first curve functions in the first surface region R1 may be correspondingly obtained. As such, the second strain distribution value of the second surface region R2 is further constructed according to a strain distribution corresponding to each of the first curve functions. Note that since the curve CVa shown in FIG. 6 is assumed to be a curve on the second surface region R2, the orthogonal projection C′ of the force center C on the second surface region R2 is depicted to be located on the curve CVa in FIG. 6.

Further, the data processing apparatus 120 may define a reference plane RP according to an initial state of the compressed object 50 and the maximum compressibility of the compressed object 50 pre-measured by the measurement apparatus 130. The reference plane RP has an upper side and a lower side opposite to each other. The compressing object 60 is located at the upper side of the reference plane RP. The reference plane RP is a limit position to which a surface of the compressed object 50 may move downward when being compressed down. At least one section CVa1 (i.e., a section extending from the second position P2a to an intersection point Pra between the curve CVa and the reference plane RP) of the curve CVa corresponding to the first curve function is located at the upper side of the reference plane RP as shown in FIG. 6. The data processing apparatus 120 obtains the corresponding strain distribution based on the section CVa1 of the curve CVa and the reference plane RP. Accordingly, part of the calculated strain values are prevented from being excessively large because the maximum compressibility of the compressed object 50 is not considered.

Another measurement method provided by the compression state measuring system 100 of the present embodiment is described as follows.

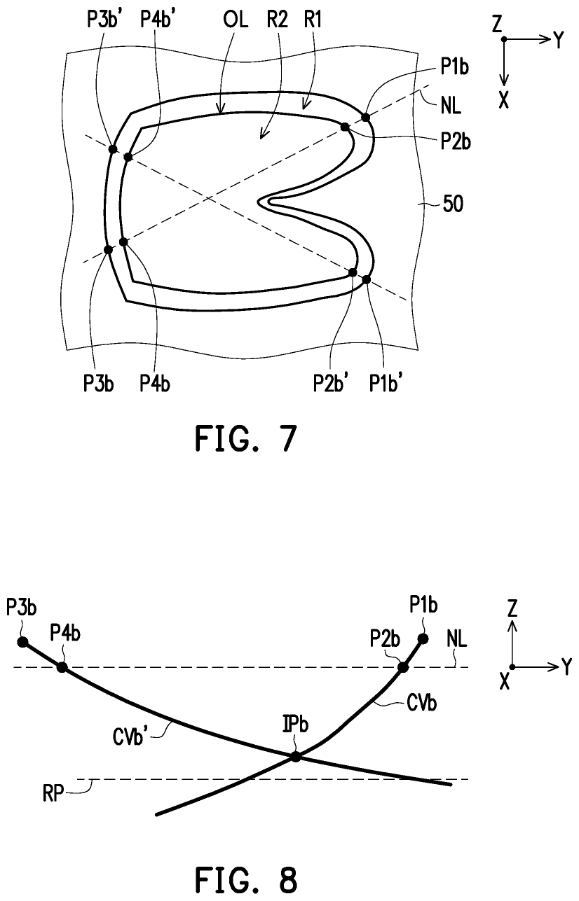

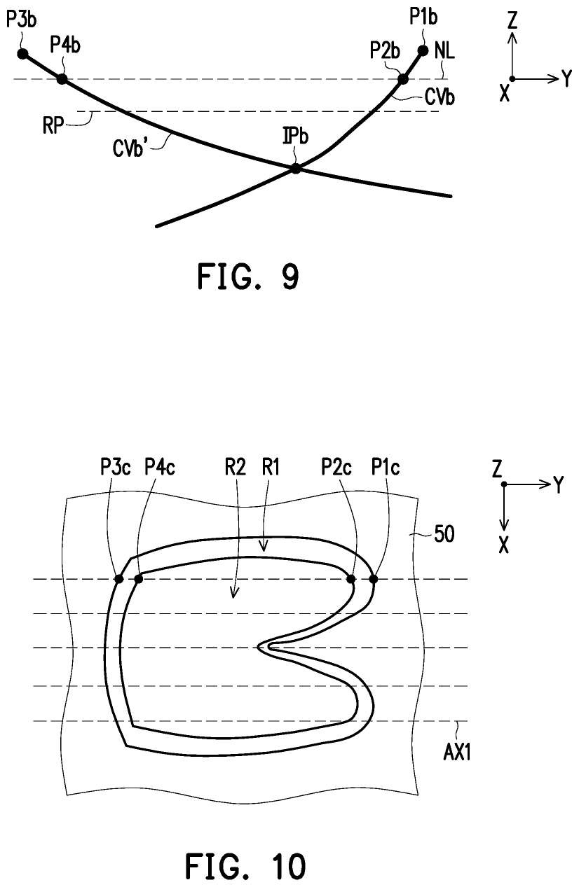

FIG. 7 is a schematic top view of the compressed object of FIG. 2B. FIG. 8 and FIG. 9 are curves schematically illustrating the compressed object of FIG. 7. The data processing apparatus 120 treats any position on the border contour OL as a second position P2b. The border contour OL has a normal line NL at the second position P2b. The data processing apparatus 120 treats any position of an orthogonal projection of the normal line NL on the first surface region R1 as a first position P1b and may obtain the corresponding first curve function according to a strain distribution between the first position P1b and the second position P2b in the first surface region R1. This first curve function is part of the strain distribution function. The strain distribution function corresponds to the curved surface formed by the first surface region R1, and the first curve function corresponds to a curve CVb (shown in FIG. 8) extending between the first position P1b and the second position P2b on the curved surface.

As described above, the data processing apparatus 120 treats another two positions of the orthogonal projection of the normal line NL on the first surface region R1 as a third position P3b and a fourth position P4b and obtain a corresponding second curve function according to a strain distribution between the third position P3b and the fourth position P4b. This second curve function is part of the strain distribution function as well. The strain distribution function corresponds to the curved surface formed by the first surface region R1, and the second curve function corresponds to a curve CVb′ (shown in FIG. 8) extending between the third position P3b and the fourth position P4b on the curved surface. Next, the data processing apparatus 120 treats the first curve function and the second curve function as a continuous deformation state extending from the first surface region R1 to the second surface region R2 and deduces a strain value in the second surface region R2 according to an intersection point IPB of the curve CVb corresponding to the first curve function and the curve CVb′ corresponding to the second curve function. In this way, a plurality of different first positions P1b (another first position P1b′ and a corresponding second position P2b′ and another third P3b′ and a corresponding fourth position P4b′ are schematically illustrated) are continuously selected in the first surface region R1, so that a plurality of different first curve functions and a plurality of different corresponding second curve functions in the first surface region R1 may be correspondingly obtained. As such, a strain distribution value in the second surface region R2 is constructed according to an intersection point of the curve corresponding to the first curve function and the curve corresponding to the second curve function.

Further, the data processing apparatus 120 may define a reference plane RP according to the initial state of the compressed object 50 and the maximum compressibility of the compressed object 50 pre-measured by the measurement apparatus 130. The reference plane RP is a limit position to which a surface of the compressed object 50 may move downward when being compressed down. If the intersection point IPb of the curve CVb corresponding to the first curve function and the curve CVb′ corresponding to the second curve function is located at the upper side of the reference plane RP as shown in FIG. 8, the data processing apparatus 120 obtains the strain value in the second surface region R2 based on the intersection point IPb of the curve CVb corresponding to the first curve function and the curve CVb′ corresponding to the second curve function as described above. In contrast, if the intersection point IPb of the curve CVb corresponding to the first curve function and the curve CVb′ corresponding to the second curve function is not located at the upper side of the reference plane RP as shown in FIG. 9, the data processing apparatus 120 obtains the strain value in the second surface region R2 based on the reference plane RP instead. Accordingly, part of the calculated strain values are prevented from being excessively large because the maximum compressibility of the compressed object 50 is not considered.

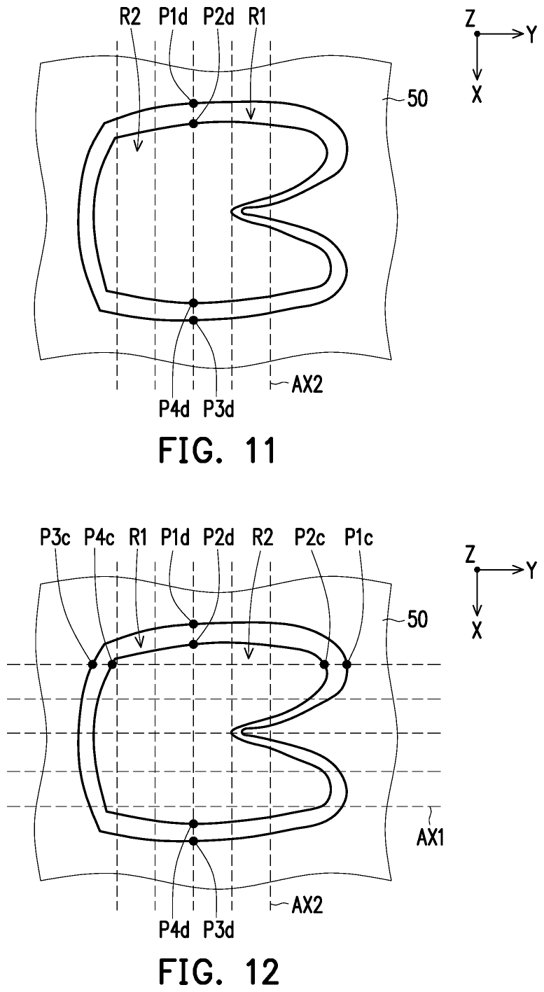

Compared to the embodiments shown FIG. 7 to FIG. 9, in other embodiments, each of the first position and the second position and the corresponding third position and the fourth position may be determined according to other appropriate manners. Examples are described through drawings as follows. FIG. 10 to FIG. 13 are schematic top views of the compressed object of FIG. 2B. In the embodiment shown in FIG. 10, in the data processing apparatus 120, a plurality axis lines AX1 parallel to one another and extending horizontally replace the normal line NL shown in FIG. 7 to accordingly define the first position and the second position and the corresponding third position and the fourth position (one group of a first position Plc, a second position P2c, a third position P3c, and a fourth position P4c are schematically marked in FIG. 10). In the embodiment shown in FIG. 11, in the data processing apparatus 120, a plurality axis lines AX2 parallel to one another and extending vertically replace the normal line NL shown in FIG. 7 to accordingly define the first position and the second position and the corresponding third position and the fourth position (one group of a first position P1d, a second position P2d, a third position P3d, and a fourth position P4d are schematically marked in FIG. 11). In the embodiment shown in FIG. 12, in the data processing apparatus 120, the plurality axis lines AX1 parallel to one another and extending horizontally and the plurality of axis lines AX2 parallel to one another and extending vertically replace the normal line NL shown in FIG. 7 to accordingly define the first position and the second position and the corresponding third position and the fourth position (one group of the first position Plc, the second position P2c, the third position P3c, and the fourth position P4c and the other group of the first position P1d, the second position P2d, the third position P3d, and the fourth position P4d are schematically marked in FIG. 11). In the embodiment shown in FIG. 13, in the data processing apparatus 120, a plurality axis lines AX3 parallel to one another and extending diagonally replace the normal line NL shown in FIG. 7 to accordingly define the first position and the second position and the corresponding third position and the fourth position (one group of a first position P1e, a second position P2e, a third position P3e, and a fourth position P4e are schematically marked in FIG. 13).

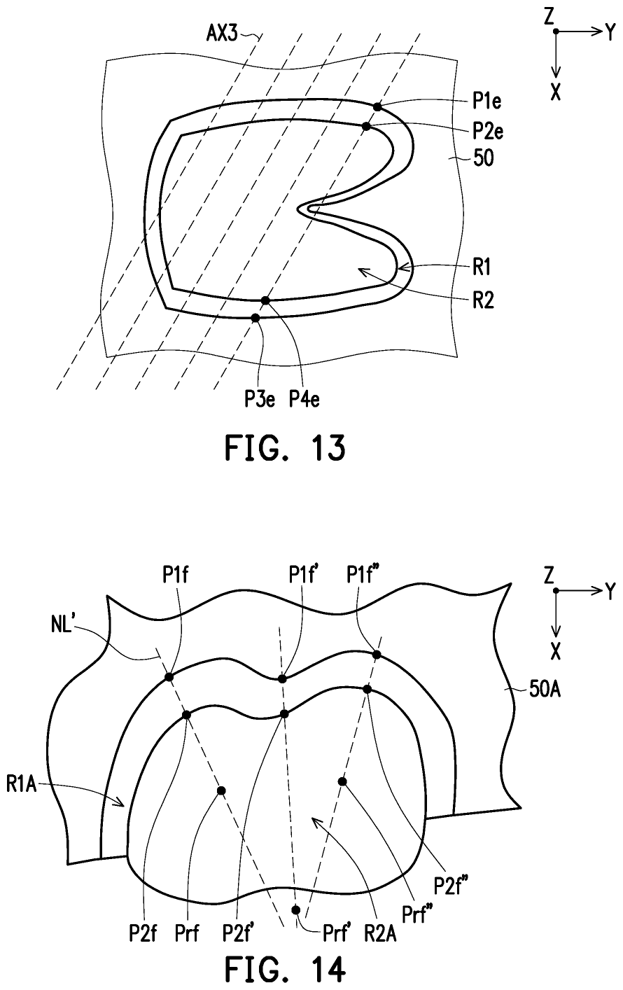

FIG. 14 is a schematic partial top view of a compressed object according to another embodiment of the disclosure. In the embodiment shown in FIG. 14, the image capturing apparatus 110 (shown in FIG. 1) merely captures an image of a partial first surface region R1A of a compressed object 50A. In this case, a plurality of groups of first positions and second positions (first positions P1f, P1f′, and P1f″ and second positions P2f, P2f′, and P2f″ are schematically shown) and the corresponding normal line NL′ may be determined in the embodiment of FIG. 14 through the same manner adopted in the embodiment of FIG. 7, and a strain distribution of the second position R2A may be constructed based on a plurality of corresponding curve functions. Further, as shown in the embodiment of FIG. 6, the constructed strain distribution may be corrected according to a reference plane (such as the reference plane RP shown in FIG. 6) corresponding to a maximum compressibility of the compressed object 50A. To be specific, a manner used to define a plurality of intersection points Prf, Prf′, and Prf″ shown in FIG. 14 is identical to that used to define the intersection point Pra shown in FIG. 14, that is, the intersection points are the intersections points of a plurality of curve functions and the reference plane. Herein, the intersection points Prf and Prf″ corresponding to the second positions P2f and P2f″ are located in the second surface region R2A. As such, a corresponding strain distribution may be obtained based on a curve extending from the second position P2f to the intersection point Prf, and a corresponding strain distribution may be obtained based on a curve extending from the second position P2f″ to the intersection point Prf″. The intersection point Prf′ corresponding to the second position P2f′ is located outside the second surface region R2A, so that the corresponding strain distribution is obtained based on a curve extending from the second position P2f′ to a boundary of the second surface region R2A.

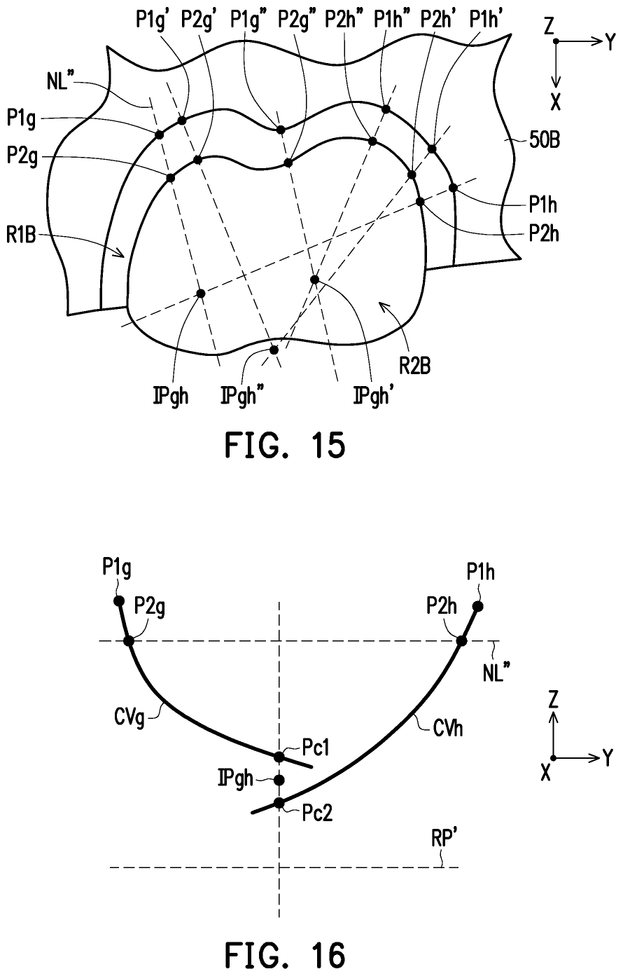

FIG. 15 is a schematic partial top view of a compressed object according to another embodiment of the disclosure. FIG. 16 is a curve schematically illustrating the compressed object of FIG. 15. In the embodiment shown in FIG. 15, the image capturing apparatus 110 (shown in FIG. 1) merely captures an image of a partial first surface region R1B of a compressed object 50B. In this case, a plurality of groups of first positions and second positions (first positions P1g, P1g′, P1g″, P1h, P1h′, and P1h″ and second positions P2g, P2g′, P2g″, P2h, P2h′, and P2h″ are schematically shown) and a corresponding normal line NL″ may be determined in the embodiment of FIG. 15 through the same manner adopted in the embodiment of FIG. 7. Two corresponding positions Pc1 and Pc2 (shown in FIG. 16) on two corresponding curves CVg and CVh (shown in FIG. 16) may be obtained based on intersection points (intersection points IPgh, IPgh′, and IPgh″ are schematically shown in FIG. 15) of orthogonal projections of any two normal lines NL″ on the second surface region R2B. Further, a strain value in the second surface region R2B is obtained according to the two corresponding positions Pc1 and Pc2. A plurality of strain values obtained in this way may be used to construct a strain distribution of the second surface region R2B. Note that since the curve CVh and the curve CVg shown in FIG. 16 are intended to be used to deduce a curve on the second surface region R2B, the intersection point IPgh of the orthogonal projection of any two normal lines NL″ on the second surface region R2B are illustrated to be located between the curve CVh and the curve CVg.

Further, the data processing apparatus 120 may define a reference plane RP′ (shown in FIG. 16) according to the initial state of the compressed object 50 and the maximum compressibility of the compressed object 50 pre-measured by the measurement apparatus 130. The reference plane RP′ is a limit position to which a surface of the compressed object 50B may move downward when being compressed down. If the two corresponding positions Pc1 and Pc2 are located at an upper side of the reference plane RP′ as shown in FIG. 16, the data processing apparatus 120 obtains a strain value in the second surface region R2B based on one of or a median value of the two corresponding positions Pc1 and Pc2. In contract, if the two corresponding positions Pc1 and Pc2 are not located at the upper side of the reference plane RP′, the data processing apparatus 120 obtains the strain value in the second surface region R2B based on the reference plane RP′ instead. Accordingly, part of the calculated strain values are prevented from being excessively large because the maximum compressibility of the compressed object 50 is not considered. Besides, if the intersection point IPgh″ shown in FIG. 15 is located outside the second surface region R2B, the corresponding strain distribution is obtained based on a section of a corresponding curve in the second surface region R2B and the reference plane RP′, and a specific manner used herein may be similar to that provided in the embodiment of FIG. 14.

In view of the foregoing, the embodiments of the disclosure provide a compression state measuring method and a compression state measuring system through which a compressed state of a surface region of a compressed object covered by a compressing object may be obtained in real time. In the disclosure, the strain distribution function corresponding to the first surface region is calculated through the image of the first surface region of the compressed object which is not covered. Further, based on a certain degree of continuity characteristic of the deformation state of the compressed object when being pressed, the strain distribution function may be treated as the deformation state corresponding to the overall compressed surface of the compressed object. Therefore, in the case that the image of the covered second surface region of the compressed object is not obtained, the strain distribution value of the second surface region may be calculated in real time through the strain distribution function, and the stress distribution value of the second surface region may thus be further calculated in real time.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A compression state measuring method, adapted to measure a compressed state of a compressed object compressed by a compressing object, the compression state measuring method comprising:
capturing at least one image of a first surface region of the compressed object not covered by the compressing object;
obtaining a first strain distribution value of the first surface region according to the at least one image;
obtaining at least one strain distribution function according to the first strain distribution value; and
obtaining a second strain distribution value of a second surface region of the compressed object covered by the compressing object according to the at least one strain distribution function.

2. The compression state measuring method according to claim 1, further comprising:
obtaining a corresponding stress distribution value according to the second strain distribution value and an elastic modulus of the compressed object.

3. The compression state measuring method according to claim 1, wherein the step of obtaining the second strain distribution value of the second surface region of the compressed object covered by the compressing object according to the at least one strain distribution function comprises:
measuring a maximum compressibility of the compressed object; and
obtaining the second strain distribution value according to the at least one strain distribution function and the maximum compressibility.

4. The compression state measuring method according to claim 1, wherein a number of the at least one image is plural, and the images respectively correspond to different time points or correspond to different angles of shot.

5. The compression state measuring method according to claim 1, wherein the at least one strain distribution function comprises a first curve function.

6. The compression state measuring method according to claim 5, wherein the step of obtaining the at least one strain distribution function comprises:
obtaining the first curve function according to a strain distribution between a first position and a second position in the first surface region.

7. The compression state measuring method according to claim 6, wherein a border contour is provided between the first surface region and the second surface region, and the step of determining the first position and the second position comprises:
obtaining a force center of the compressed object according to the first strain distribution value;
treating any position in the first surface region as the first position, wherein a connection line is provided between the force center and the first position; and
treating an intersection point of an orthogonal projection of the connection line in the first surface region and the second surface region and the border contour as the second position.

8. The compression state measuring method according to claim 7, wherein the step of obtaining the second strain distribution value comprises:
obtaining a strain distribution between an orthogonal projection of the force center in the second surface region and the second position according to the first curve function.

9. The compression state measuring method according to claim 8, wherein the step of obtaining the strain distribution between the orthogonal projection of the force center in the second surface region and the second position according to the first curve function comprises:
measuring a maximum compressibility of the compressed object;
defining a reference plane according to an initial state of the compressed object and the maximum compressibility, wherein the reference plane has an upper side and a lower side opposite to each other, the compressing object is located at the upper side of the reference plane, and at least one section of a curve corresponding to the first curve function is located at the upper side of the reference plane; and
obtaining a corresponding strain distribution based on the at least one section of the curve corresponding to the first curve function and the reference plane.

10. The compression state measuring method according to claim 8, comprising:
obtaining a plurality of different first curve functions; and
constructing the second strain distribution value according to a strain distribution corresponding to each of the first curve functions.

11. The compression state measuring method according to claim 6, wherein a border contour is provided between the first surface region and the second surface region, and the step of determining the first position and the second position comprises:
treating any position on the border contour as the second position, wherein the border contour has a normal line at the second position; and
treating any position of an orthogonal projection of the normal line on the first surface region as the first position.

12. The compression state measuring method according to claim 11, further comprising:
treating another two positions of the orthogonal projection of the normal line on the first surface region as a third position and a fourth position;
obtaining a second curve function according to a strain distribution between the third position and the fourth position; and
obtaining a strain value in the second surface region according to an intersection point of a curve corresponding to the first curve function and a curve corresponding to the second curve function.

13. The compression state measuring method according to claim 12, wherein the step of obtaining the strain value in the second surface region according to the intersection point of the curve corresponding to the first curve function and the curve corresponding to the second curve function comprises:
measuring a maximum compressibility of the compressed object;
defining a reference plane according to an initial state of the compressed object and the maximum compressibility, wherein the reference plane has an upper side and a lower side opposite to each other, the compressing object is located at the upper side of the reference plane;
obtaining the strain value in the second surface region based on the intersection point of the curve corresponding to the first curve function and the curve corresponding to the second curve function if the intersection point of the curve corresponding to the first curve function and the curve corresponding to the second curve function is located at the upper side; and
obtaining the strain value in the second surface region based on the reference plane if the intersection point of the curve corresponding to the first curve function and the curve corresponding to the second curve function is not located at the upper side of the reference plane.

14. The compression state measuring method according to claim 13, comprising:
obtaining a plurality of different first curve functions and a plurality of different corresponding second curve functions; and
constructing the second strain distribution value based on an intersection point of a curve corresponding to each of the first curve functions and a curve corresponding to the corresponding second curve function.

15. The compression state measuring method according to claim 11, wherein the step of obtaining the at least one strain distribution function comprises:
obtaining at least two first curve functions;
respectively obtaining two corresponding positions on curves corresponding to the at least two first curve functions according to an intersection point of orthogonal projections of two normal lines corresponding to the at least two first curve functions on the second surface region; and
obtaining a strain value in the second surface region according to the two corresponding positions.

16. The compression state measuring method according to claim 15, wherein the step of obtaining the strain value in the second surface region according to the two corresponding positions comprises:
measuring a maximum compressibility of the compressed object;
defining a reference plane according to an initial state of the compressed object and the maximum compressibility, wherein the reference plane has an upper side and a lower side opposite to each other, and the compressing object is located at the upper side of the reference plane;
obtaining the strain value in the second surface region based on at least one of the corresponding positions if the at least one of the corresponding positions is located at the upper side of the reference plane; and
obtaining the strain value in the second surface region based on the reference plane if the two corresponding positions are not located at the upper side of the reference plane.

17. A compression state measuring system, adapted to measure a compressed state of a compressed object compressed by a compressing object, the compression state measuring system comprising:
at least one image capturing apparatus, adapted to capture at least one image of a first surface region of the compressed object not covered by the compressing object; and
a data processing apparatus, adapted to obtain a first strain distribution value of the first surface region according to the at least one image, adapted to obtain at least one strain distribution function according to the first strain distribution value, adapted to obtain a second strain distribution value of a second surface region of the compressed object covered by the compressing object according to the at least one strain distribution function.

18. The compression state measuring system according to claim 17, wherein the data processing apparatus is adapted to obtain a corresponding stress distribution value according to the second strain distribution value and an elastic modulus of the compressed object.

19. The compression state measuring system according to claim 17, further comprising a measurement apparatus, wherein the measurement apparatus is adapted to measure a maximum compressibility of the compressed object, and the data processing apparatus is adapted to obtain the second strain distribution value according to the at least one strain distribution function and the maximum compressibility.

20. The compression state measuring system according to claim 17, wherein a number of the at least one image is plural, and the images respectively correspond to different time points or correspond to different angles of shot.

21. The compression state measuring system according to claim 17, wherein the at least one strain distribution function comprises a first curve function.

22. The compression state measuring system according to claim 21, wherein the data processing apparatus is adapted to obtain the first curve function according to a strain distribution between a first position and a second position in the first surface region.

23. The compression state measuring system according to claim 22, wherein a border contour is provided between the first surface region and the second surface region, and the data processing apparatus is adapted to obtain a force center of the compressed object according to the first strain distribution value and is adapted to treat any position in the first surface region as the first position, wherein a connection line is provided between the force center and the first position, and the data processing apparatus is adapted to treat an intersection point of an orthogonal projection of the connection line in the first surface region and the second surface region and the border contour as the second position.

24. The compression state measuring system according to claim 23, wherein the data processing apparatus is adapted to obtain a strain distribution between an orthogonal projection of the force center in the second surface region and the second position according to the first curve function.

25. The compression state measuring system according to claim 24, further comprising a measurement apparatus, wherein the measurement apparatus is adapted to measure a maximum compressibility of the compressed object, and the data processing apparatus is adapted to define a reference plane according to an initial state of the compressed object and the maximum compressibility, wherein the reference plane has an upper side and a lower side opposite to each other, the compressing object is located at the upper side of the reference plane, at least one section of a curve corresponding to the first curve function is located at the upper side of the reference plane, and the data processing apparatus is adapted to obtain a corresponding strain distribution based on the at least one section of the curve corresponding to the first curve function and the reference plane.

26. The compression state measuring system according to claim 24, wherein the data processing apparatus is adapted to obtain a plurality of different first curve functions and is adapted to construct the second strain distribution value according to a strain distribution corresponding to each of the first curve functions.

27. The compression state measuring system according to claim 22, wherein a border contour is provided between the first surface region and the second surface region, and the data processing apparatus is adapted to treat any position on the border contour as the second position, wherein the border contour has a normal line at the second position, and the data processing apparatus is adapted to treat any position of an orthogonal projection of the normal line on the first surface region as the first position.

28. The compression state measuring system according to claim 27, wherein the data processing apparatus is adapted to treat another two positions of the orthogonal projection of the normal line on the first surface region as a third position and a fourth position, is adapted to obtain a second curve function according to a strain distribution between the third position and the fourth position, and is adapted to obtain a strain value in the second surface region according to an intersection point of a curve corresponding to the first curve function and a curve corresponding to the second curve function.

29. The compression state measuring system according to claim 28, further comprising a measurement apparatus, wherein the measurement apparatus is adapted to measure a maximum compressibility of the compressed object, the data processing apparatus is adapted to define a reference plane according to an initial state of the compressed object and the maximum compressibility, the reference plane has an upper side and a lower side opposite to each other, the compressing object is located at the upper side of the reference plane, the data processing apparatus obtains the strain value in the second surface region based on the intersection point of the curve corresponding to the first curve function and the curve corresponding to the second curve function if the intersection point of the curve corresponding to the first curve function and the curve corresponding to the second curve function is located at the upper side of the reference plane, and the data processing apparatus obtains the strain value in the second surface region based on the reference plane if the intersection point of the curve corresponding to the first curve function and the curve corresponding to the second curve function is not located at the upper side of the reference plane.

30. The compression state measuring system according to claim 29, wherein the data processing apparatus is adapted to obtain a plurality of different first curve functions and a plurality of different corresponding second curve functions and is adapted to construct the second strain distribution value based on an intersection point of a curve corresponding to each of the first curve functions and a curve corresponding to the corresponding second curve function.

31. The compression state measuring system according to claim 27, wherein the data processing apparatus is adapted to obtain at least two first curve functions, is adapted to respectively obtain two corresponding positions on curves corresponding to the at least two first curve functions according to an intersection point of orthogonal projections of two normal lines corresponding to the at least two first curve functions on the second surface region, and is adapted to obtain a strain value in the second surface region according to the two corresponding positions.

32. The compression state measuring system according to claim 31, further comprising a measurement apparatus, wherein the measurement apparatus is adapted to measure a maximum compressibility of the compressed object, the data processing apparatus is adapted to define a reference plane according to an initial state of the compressed object and the maximum compressibility, the reference plane has an upper side and a lower side opposite to each other, the compressing object is located at the upper side of the reference plane, the data processing apparatus obtains the strain value in the second surface region based on at least one of the corresponding positions if the at least one of corresponding positions is located at the upper side of the reference plane, and the data processing apparatus obtains the strain value in the second surface region based on the reference plane if the two corresponding positions are not located at the upper side of the reference plane.
